(19) United States
(12) Patent Application Publication
(10) Pub. No.: US 2020/0315981 A1
(43) Pub. Date: Oct. 8, 2020

(54) COMPOSITIONS AND METHODS FOR NANOPARTICLE-BASED DRUG DELIVERY AND IMAGING

(71) Applicant: Memorial Sloan Kettering Cancer Center, New York, NY (US)

(72) Inventors: Jan GRIMM, New York, NY (US); Charalambos KAITTANIS, New York, NY (US); Travis M. SHAFFER, New York, NY (US)

(73) Assignee: Memorial Sloan Kettering Cancer Center, New York, NY (US)

(21) Appl. No.: 16/659,343

(22) Filed: Oct. 21, 2019

Related U.S. Application Data
(63) Continuation of application No. 15/551,751, filed on Aug. 17, 2017, now abandoned.
(60) Provisional application No. 62/117,884, filed on Feb. 18, 2015.

(51) Int. Cl. A61K 9/51

(52) U.S. Cl. CPC A61K 9/5123; A61K 31/195; Y02A 50/411; A61K 9/5161

(57) ABSTRACT

Methods and compositions described herein use polysaccharide nanoparticles (or polysaccharide-coated nano -particles) to retain and deliver unaltered therapeutic agents to sites of disease. The polysaccharide nanoparticles are non-covalently associated with the unaltered therapeutic agent. The polysaccharide is able to retain cargo (drugs, diagnostics, etc.) without chemical modification of the agent. The nanoparticle maintains its association with the agent through non-covalent interactions but releases its agent in response to changes in the microenvironment, e.g., at the site of cancer cells or cancer tissue.

### CROSS-REFERENCE TO RELATED APPLICATIONS

This application is a continuation of U.S. patent application Ser. No. 15/551,751, filed Aug. 17, 2017, which is a National Stage Application of PCT/US2016/018240, filed Feb. 17, 2016, which claims the benefit of and priority to U.S. Provisional Patent Application No. 62/117,884, filed Feb. 18, 2015, the entire contents of each of which are incorporated herein by reference.

### FIELD OF THE INVENTION

This invention relates generally to nanoparticles as agents of drug and diagnostic delivery to tissue targets. More particularly, in certain embodiments, polysaccharide nanoparticles for delivery of unaltered therapeutic agents and retention of radiological tracers are described herein.

### BACKGROUND

Effective treatment relies on the successful delivery of therapeutics to the site of the disease. Particularly for cancer chemotherapy, the drug or combination of drugs has to reach the tumor at concentrations sufficient to cause tumor regression, to prevent the emergence of drug resistant cell populations, and to avert metastatic niche. However, the toxicity of many such drugs restricts dosages that may be used safely. It is desirable that these compounds stay in circulation for sufficient time in order to reach the pathology, without affecting healthy organs and tissue and avoiding clearance from the kidneys or liver.

Nanotechnology has addressed this urgent clinical need with the introduction of novel drug delivery platforms, including liposomes and polymeric nanoparticles, which are either in the clinic or ongoing clinical trials. For example, the liposomal formulation Doxil (Doxorubicin) and AmBisome (Amphotericin B) offer enhanced pharmacokinetics and high drug delivery of compounds with poor aqueous solubility. The drug release mechanism of these vehicles relies on either plasma membrane fusion or the enzymatic activity of lipases, which may cause side effects and hepatic toxicity. In the case of polymeric nanoparticles that are constructed with polymers such as poly(lactic-co-glycolic) acid (PLGA) and hydrophobic-core polyesters (HBPE), the therapeutic cargo is released when the polymer undergoes acid hydrolysis, usually in the late endosomal and lysosomal compartments, or in the presence of lytic enzymes, like esterases.

Recently, it was postulated that carboxymethyl dextran-coated iron oxide nanoparticles (Ferumoxytol, Feraheme®) could serve as a drug delivery system. It is presently found that the carboxymethyl dextran coating of the nanoparticles appears to retain diverse therapeutic payloads via weak electrostatic interactions, which, once perturbed, such as by mild acidification of their microenvironment or local elevation of the ionic strength, rapidly releases their cargo. However, the use of an iron oxide nanoparticle-based system may have inefficient cargo retention and the potential for iron toxicity, in certain instances.

Acknowledging that cancer is a heterogeneous disease that frequently requires chemotherapy with a combination of drugs, other facile drug delivery carriers that are amenable to rapid transition from the lab to the patient bedside would be useful in the treatment of heterogeneous diseases such as cancer. Among the drawbacks of current nanoparticle-based drug delivery platforms is the requirement that the drug be chemically modified to allow covalent attachment to the nanoparticle, which may adversely affect drug activity.

### SUMMARY OF THE INVENTION

Presented herein are methods and compositions for delivering therapeutics and/or imaging agents to sites of disease in a subject by non-covalently associating the agent with polysaccharide nanoparticles. These drug delivery platforms do not require chemical modification of the payload for attachment to the nanoparticle, nor is chemical modification of the nanoparticle required. Thus, the regulatory approval process may be less time consuming and less expensive for these drug delivery platforms.

For example, following administration of the composition to a subject, the therapeutic payload is delivered (inactive) to a cancer site, then the drug is released (rendered active) in the solid cancer microenvironment, taking advantage of the tumor's aberrant metabolism and enhanced glycolytic activity that lowers the stromal and interstitial pH. Spatiotemporal drug release can be monitored via MRI, further facilitating individualized dosing of therapeutics for more effective treatment with less severe, reduced, or eliminated side effects. Moreover, the platforms described herein do not have the drawbacks of some iron oxide nanoparticle-based systems. For example, in some instances, iron oxide nanoparticle-based system may have inefficient cargo retention and risk of iron toxicity.

Thus, in certain embodiments, methods and compositions are provided herein that use polysaccharide nanoparticles (or polysaccharide-coated nanoparticles) to retain and deliver unaltered therapeutic agents to sites of disease. The polysaccharide nanoparticle is non-covalently associated with the unaltered therapeutic agent. The polysaccharide is able to retain cargo (drugs, diagnostics, etc.) without chemical modification of the agent. The nanoparticle maintains its association with the agent through non-covalent interactions but releases its agent in response to changes in the microenvironment, e.g., at the site of cancer cells or cancer tissue.

In one aspect, the invention is directed to a method of delivering one or more agents (e.g., therapeutic agent, imaging agent) to a site (e.g., a disease site, infection site, inflammation site, or organ) in a subject (e.g., suffering from or susceptible to a disease, disorder, or condition), the method comprising: administering a composition (e.g., pharmaceutical composition) comprising: one or more unaltered agents (e.g., unaltered therapeutic agents and/or unaltered imaging agents) associated with nanoparticles comprising (e.g., comprising, consisting of, or consisting essentially of) a polysaccharide (e.g., wherein a discrete unaltered agent molecule is associated with a discrete nanoparticle).

In certain embodiments, at least a medically effective portion of the one or more unaltered agents (e.g., without chemical modification) maintains non-covalent (e.g., weak electrostatic, hydrogen bond, van der Waals) interactions with the nanoparticles in the subject upon administration to the subject up until arrival of the one or more agents (therapeutic agents, imaging agents) at the site in the subject (e.g., disease site, infection site, inflammation site), whereby a retained portion of the agents is inactive while being retained by the nanoparticles, and whereby a microenvironment (e.g., acidity, osmolarity, ionic strength, hypoxia) at the site causes release of at least a medically effective portion of the one or more unaltered agents from the nanoparticles at the site.

In certain embodiments, the nanoparticles are at least 50 wt. % polysaccharide (e.g., at least 60 wt. %, at least 70 wt. %, at least 80 wt. %, at least 90 wt. %).

In certain embodiments, each of the nanoparticles have a surface comprising the polysaccharide.

In certain embodiments, the nanoparticles have an average diameter within a range of 1 nm-500 nm (e.g., 1 nm-10 nm, 10 nm-25 nm, 25 nm-50 nm, 50 nm-100 nm, or 100 nm-500 nm).

In certain embodiments, the polysaccharide has a molecular weight within a range of 1 kDa to 1 million kDa (e.g., 1 kDa-10 kDa, 10 kDa-100 kDa, 100 kDa-1000 kDa, or 1000 kDa-1,000,000 kDa).

In certain embodiments, the polysaccharide comprises a member selected from the group consisting of dextran, amylose, amylopectin, glycogen, cellulose, arabonixylan, and pectin.

In certain embodiments, the disease, disorder, or condition is a member selected from the group consisting of cancer, rheumatoid arthritis, atherosclerosis, cystic fibrosis, diabetic ketoacidosis, cardiac arrest, stroke, renal failure, malaria, lactic acid acidosis, and inflammation.

In certain embodiments, the disease, disorder, or condition is cancer.

In certain embodiments, the cancer is a member selected from the group consisting of prostate cancer, breast cancer, brain cancer, testicular cancer, cervical cancer, lung cancer, colon cancer, glioma, glioblastoma, multiple myeloma, sarcoma, bone cancer, small cell carcinoma, renal cancer, liver cancer, head and neck cancer, esophageal cancer, thyroid cancer, lymphoma, and leukemia.

In certain embodiments, the unaltered therapeutic is a chemotherapy drug.

In certain embodiments, the chemotherapy drug is a member selected from the group consisting of doxorubicin, amphotericin B, daunarubicine, cytarabine, enzalutamide, methotrexate, cytarabine, gemcitabine, decitabine, azacitidine, fludarabine, nelarabine, cladribine, clofarabine, pentostatin, thioguanine, mercaptopurine, photosensitizer (e.g., photodynamic therapy agent), biologic, including peptides and peptidomimetics, and kinase inhibitor.

In certain embodiments, the chemotherapeutic drug is doxorubicin.

In certain embodiments, the unaltered agent is sufficiently hydrophobic such that it is insoluble or only partly (e.g., sparingly) soluble in water and/or an aqueous buffer solution, but is soluble in an organic solvent (e.g., a water-immiscible and/or water-miscible organic solvent) (e.g., DMSO, DMF, etc.).

In certain embodiments, the agent is an imaging agent (e.g., its presence, release, and/or both in the subject following administration can be monitored via an imaging system).

In certain embodiments, the composition comprises at least one therapeutic agent and at least one imaging agent (e.g., wherein the imaging agent is a member selected from the group consisting of a fluorophore, a pigment/dye, a contrast agent, a radionuclide and a PET tracer) associated with the nanoparticles.

In certain embodiments, the polysaccharide is a dextran (e.g., substituted or unsubstituted, e.g., dextran, carboxymethyl dextran, etc.).

In certain embodiments, the nanoparticles have an average diameter of at least 5 nm (e.g., as loaded with the one or more unaltered therapeutics as measured in a physiologically relevant solution) (e.g., at least 10 nm, e.g., at least 15 nm). 100281 In certain embodiments, the nanoparticles have an average diameter between 15 nm and 200 nm.

In certain embodiments, the nanoparticles do not have a crystalline core (e.g., a metal core, a metal oxide core, a metalloid (e.g., iron, gold, silver, cerium, gadolinium) core, or a metalloid oxide core).

In certain embodiments, the non-covalent (weak) interactions comprise electrostatic interactions between polysaccharide functional groups and functional groups (e.g., side chains) of the one or more unaltered therapeutic agents.

In certain embodiments, the composition further comprises an excipient.

In another aspect, the invention is directed to a composition (e.g., pharmaceutical composition), comprising: one or more unaltered agents (e.g., therapeutic agent, imaging agent) associated with nanoparticles comprising (e.g., comprising, consisting of, or consisting essentially of) a polysaccharide (e.g., wherein a discrete unaltered agent molecule is associated with a discrete nanoparticle).

In certain embodiments, at least a medically effective portion of the one or more unaltered (e.g., without chemical modification) agents (e.g., therapeutic agent, imaging agents) maintains non-covalent (e.g., weak electrostatic, hydrogen bond, van der Waals) interactions with the nanoparticles in a first environment (e.g., in the container, in blood); and wherein in a second environment (e.g., site of action with a different acidity, osmolarity, ionic strength, hypoxia), at least a medically effective portion of the one or more unaltered agents is released from (e.g., is no longer associated with) the nanoparticles.

In certain embodiments, the unaltered agent is a chemotherapy drug.

In certain embodiments, the chemotherapy drug is a member selected from the group consisting of doxorubicin, amphotericin B, daunarubicine, cytarabine, enzalutamide, methotrexate, cytarabine, gemcitabine, decitabine, azacitidine, fludarabine, nelarabine, cladribine, clofarabine, pentostatin, thioguanine, mercaptopurine, photosensitizer (photodynamic therapy agent), biologic, including peptides and peptidomimetics, kinase inhibitors, and combinations thereof.

In certain embodiments, the chemotherapy drug is doxorubicin.

In certain embodiments, the unaltered therapeutic is sufficiently hydrophobic such that it is insoluble or only partly (e.g., sparingly) soluble in water and/or an aqueous buffer solution, but is soluble in an organic solvent (e.g., a water-immiscible and/or water-miscible organic solvent) (e.g., DMSO, DMF, etc.).

In certain embodiments, the unaltered agent is an imaging agent (e.g., its presence, release, and/or both in the subject following administration can be monitored via an imaging system).

In certain embodiments, the composition comprises at least one therapeutic agent and at least one imaging agent (e.g., wherein the imaging agent is a member selected from the group consisting of a fluorophore, a pigment/dye, a contrast agent, a radionuclide and a PET tracer) associated with the nanoparticles.

In certain embodiments, the polysaccharide is a member selected from the group consisting of dextran, amylose, amylopectin, glycogen, cellulose, arabonixylan, and pectin.

In certain embodiments, the polysaccharide is a dextran (e.g., substituted or unsubstituted, e.g., dextran, carboxymethyl dextran, etc.).

In certain embodiments, the nanoparticles have an average diameter of at least 5 nm (e.g., as loaded with the one or more unaltered therapeutics as measured in a physiologically relevant solution) (e.g., at least 10 nm, e.g., at least 15 nm).

In certain embodiments, the nanoparticles have an average diameter between 15 nm and 200 nm.

In certain embodiments, the nanoparticles do not have a crystalline core (e.g., a metal core, a metal oxide core, a metalloid (e.g., iron, gold, silver, cerium, gadolinium) core, or a metalloid oxide core).

In certain embodiments, the nanoparticles do not contain iron.

In certain embodiments, the composition further comprises a carrier.

In another aspect, the invention is directed to a composition (e.g., pharmaceutical composition) comprising one or more unaltered agents (e.g., unaltered therapeutic agents, unaltered imaging agents) associated with nanoparticles comprising (e.g., comprising, consisting of, or consisting essentially of) a polysaccharide (e.g., wherein a discrete unaltered agent molecule is associated with a discrete nanoparticle) for use in a method of treating cancer in a subject (e.g., suffering from or susceptible to a disease, disorder, or condition), wherein the treating comprises delivering the composition to a site (e.g., a disease site, infection site, inflammation site, or organ) in the subject.

In another aspect, the invention is directed to a composition (e.g., pharmaceutical composition) comprising one or more unaltered agents (e.g., unaltered therapeutic agents, unaltered imaging agents) associated with nanoparticles comprising (e.g., comprising, consisting of, or consisting essentially of) a polysaccharide (e.g., wherein a discrete unaltered agent molecule is associated with a discrete nanoparticle) for use in a method of in vivo diagnosis of cancer in a subject (e.g., suffering from or susceptible to a disease, disorder, or condition), wherein the in vivo diagnosis comprises delivering the composition to a site (e.g., a disease site, infection site, inflammation site, or organ) in the subject.

In another aspect, the invention is directed to a composition (e.g., pharmaceutical composition) comprising one or more unaltered agents (e.g., unaltered therapeutic agents, unaltered imaging agents) associated with nanoparticles comprising (e.g., comprising, consisting of, or consisting essentially of) a polysaccharide (e.g., wherein a discrete unaltered agent molecule is associated with a discrete nanoparticle) for use in (a) a method of treating cancer in a subject or (b) a method of in vivo diagnosis of cancer in a subject, wherein the method comprises delivering the composition to a site (e.g., a disease site, infection site, inflammation site, or organ) in the subject.

In another aspect, the invention is directed to a composition (e.g., pharmaceutical composition) comprising one or more unaltered agents (e.g., unaltered therapeutic agents, unaltered imaging agents associated with nanoparticles comprising (e.g., comprising, consisting of, or consisting essentially of) a polysaccharide (e.g., wherein a discrete unaltered agent molecule is associated with a discrete nanoparticle) for use in therapy.

In another aspect, the invention is directed to a composition (e.g., pharmaceutical composition) comprising one or more unaltered agents (e.g., unaltered therapeutic agents, unaltered imaging agents) associated with nanoparticles comprising (e.g., comprising, consisting of, or consisting essentially of) a polysaccharide (e.g., wherein a discrete unaltered agent molecule is associated with a discrete nanoparticle) for use in in vivo diagnosis.

In certain embodiments, at least a medically effective portion of the one or more unaltered (e.g., without chemical modification) agents (e.g., therapeutic agent, imaging agents) maintains non-covalent (e.g., weak electrostatic, hydrogen bond, van der Waals) interactions with the nanoparticles in a first environment (e.g., in the container, in blood); and wherein in a second environment (e.g., site of action with a different acidity, osmolarity, ionic strength, hypoxia), at least a medically effective portion of the one or more unaltered agents is released from the nanoparticles.

In certain embodiments, at least a medically effective portion of the one or more unaltered agents (e.g., without chemical modification) maintains non-covalent (e.g., weak electrostatic, hydrogen bond, van der Waals) interactions with the nanoparticles in the subject upon administration to the subject up until arrival of the one or more agents (therapeutic agents, imaging agents) at the site in the subject (e.g., disease site, infection site, inflammation site), whereby a retained portion of the agents is inactive while being retained by the nanoparticles, and whereby a microenvironment (e.g., acidity, osmolarity, ionic strength, hypoxia) at the site causes release of at least a medically effective portion of the one or more unaltered agents from the nanoparticles at the site.

In certain embodiments, the nanoparticles are at least 50 wt. % polysaccharide (e.g., at least 60 wt. %, at least 70 wt. %, at least 80 wt. %, at least 90 wt. %).

In certain embodiments, each of the nanoparticles have a surface comprising the polysaccharide.

In certain embodiments, the nanoparticles have an average diameter within a range of 1 nm-500 nm (e.g., 1 nm-10 nm, 10 nm-25 nm, 25 nm-50 nm, 50 nm-100 nm, or 100 nm-500 nm).

In certain embodiments, the polysaccharide has a molecular weight within a range of 1 kDa to 1 million kDa (e.g., 1 kDa-10 kDa, 10 kDa-100 kDa, 100 kDa-1000 kDa, or 1000 kDa-1,000,000 kDa).

In certain embodiments, the polysaccharide comprises a member selected from the group consisting of dextran, amylose, amylopectin, glycogen, cellulose, arabonixylan, and pectin.

In certain embodiments, wherein the polysaccharide is a dextran (e.g., substituted or unsubstituted, e.g., dextran, carboxymethyl dextran, etc.).

In certain embodiments, the disease, disorder, or condition is a member selected from the group consisting of cancer, rheumatoid arthritis, atherosclerosis, cystic fibrosis, diabetic ketoacidosis, cardiac arrest, stroke, renal failure, malaria, lactic acid acidosis, and inflammation.

In certain embodiments, the disease, disorder, or condition is cancer.

In certain embodiments, the cancer is a member selected from the group consisting of prostate cancer, breast cancer, brain cancer, testicular cancer, cervical cancer, lung cancer, colon cancer, glioma, glioblastoma, multiple myeloma, sarcoma, bone cancer, small cell carcinoma, renal cancer, liver cancer, head and neck cancer, esophageal cancer, thyroid cancer, lymphoma, and leukemia.

In certain embodiments, the unaltered therapeutic is a chemotherapy drug.

In certain embodiments, the chemotherapy drug is a member selected from the group consisting of doxorubicin, amphotericin B, daunarubicine, cytarabine, Xtandi, methotrexate, cytarabine, gemcitabine, decitabine, Vidaza, fludarabine, nelarabine, cladribine, clofarabine, pentostatin, thioguanine, mercaptopurine, photosensitizer (e.g., photodynamic therapy agent), biologic, including peptides and peptidomimetics, and kinase inhibitor.

In certain embodiments, the chemotherapy drug is doxorubicin.

In certain embodiments, the unaltered agent is sufficiently hydrophobic such that it is insoluble or only partly (e g , sparingly) soluble in water and/or an aqueous buffer solution, but is soluble in an organic solvent (e.g., a water-immiscible and/or water-miscible organic solvent) (e.g., DMSO, DMF, etc.).

In certain embodiments, the agent is an imaging agent (e.g., its presence, release, and/or both in the subject following administration can be monitored via an imaging system).

In certain embodiments, the composition comprises at least one therapeutic agent and at least one imaging agent (e.g., wherein the imaging agent is a member selected from the group consisting of a fluorophore, a pigment/dye, a contrast agent, a radionuclide and a PET tracer) associated with the nanoparticles.

In certain embodiments, the nanoparticles have an average diameter of at least 5 nm (e.g., as loaded with the one or more unaltered therapeutics as measured in a physiologically relevant solution) (e.g., at least 10 nm, e.g., at least 15 nm).

In certain embodiments, the nanoparticles have an average diameter between 15 nm and 200 nm.

In certain embodiments, the nanoparticles do not have a crystalline core (e.g., a metal core, a metal oxide core, a metalloid (e.g., iron, gold, silver, cerium, gadolinium) core, or a metalloid oxide core).

In certain embodiments, the non-covalent (weak) interactions comprise electrostatic interactions between polysaccharide functional groups and functional groups (e.g., side chains) of the one or more unaltered therapeutic agents.

In certain embodiments, the composition further comprises an excipient.

In certain embodiments, the nanoparticles do not contain iron.

In certain embodiments, the composition further comprises a carrier.

Other features, objects, and advantages of the present invention are apparent in the figures, definitions, detailed description, and claims that follow. It should be understood, however, that the figures, definitions, detailed description, and claims, while indicating embodiments of the present invention, are given by way of illustration only, not limitation. Various changes and modifications within the scope of the invention will become apparent to those skilled in the art.

### BRIEF DESCRIPTION OF THE DRAWING

The Figures described below, that together make up the Drawing, are for illustration purposes only, not for limitation.

FIGS. 1A-1D depict the retention of molecular cargo and radiological tracers with dextran nanophores.

FIG. 1A depicts spectrophotometric results from determining the retention (scavenging) efficiency of dextran nanoparticles for high-molecular-weight cargo.

FIG. 1B depicts spectrophotometric results from determining the retention (scavenging) efficiency of dextran nanoparticles for radiological tracers such as gadolinium.

FIG. 1C depicts the sequestration of the positron emitter radionuclide ⁸⁹Zr.

FIG. 1D depicts the co-retention of ⁸⁹Zr and fluorophores, such as DiI, by dextran nanoparticles.

FIGS. 2A-2F depict stable cargo retention and the release of cargo based on microenvironment differences.

FIG. 2A depicts the effects of loading cargo on the size of nanoparticles.

FIG. 2B depicts the effects of loading cargo on the surface charge of nanoparticles.

FIG. 2C depicts the stability of nanoparticle fluorescence over time.

FIG. 2D depicts the stability of nanoparticle magnetic signal over time. FIGS. 2C-2D suggest that the nanoparticles described herein retain their cargo under physiological conditions.

FIG. 2E depicts the rate of release of doxorubicin cargo by nanoparticles under acidic conditions.

FIG. 2F depicts the rate of release of gadolinium cargo by nanoparticles under acidic conditions.

FIGS. 3A-3D depict improved combinatorial therapy with co-loaded drug nanophores.

FIG. 3A depicts the change in tumor volume over time after exposure to: nanoparticle, drug alone (MDV3100 (Xtandi) or BEZ235), or nanoparticles loaded with either drug.

FIG. 3B depicts the total percent change in tumor volume after exposure to: nanoparticle, drug alone (MDV3100 (Xtandi) or BEZ235), or nanoparticles loaded with either drug.

FIG. 3C depicts the percent survival of breast cancer xenograft mice treated with control, dextran nanoparticle alone, drug (doxorubicin or AZD6244-Selumetinib) alone, or dextran nanoparticle with either drug.

FIG. 3D depicts the total percent change in tumor volume of breast cancer xenograft mice treated with control, dextran nanoparticle alone, drug (doxorubicin or AZD6244 -Selumetinib) alone, or dextran nanoparticle with either drug.

FIGS. 4A-4B depict the molecular payload sequestration with clinical dextran-coated iron oxide nanoparticles (Ferumoxytol).

FIG. 4A depicts the scavenging efficiency of Ferumoxytol nanoparticles. When comparing equal scavenging concentrations of dextran and Ferumoxytol nanophores, dextran nanoparticles more effectively sequestered DiI than Ferumoxytol.

FIG. 4B depicts the effects of cargo on the magnetic signal of Ferumoxytol nanoparticles.

FIGS. 5A-5D depicts atomic force microscopy images showing the structural stability of the nanoparticles after cargo loading and release andc confirming that the nanoparticles preserved their size after release of their cargo due to conditions within the microenvironment.

FIGS. 5A and 5B depict the stability of doxorubicin loaded nanoparticles.

FIG. 5C depicts the stability of nanoparticles at pH 7.4.

FIG. 5D depicts the stability of nanoparticles at pH 6.8.

FIG. 6 depicts cytotoxicity profiles of doxorubicin-loaded dextran nanophores. Cell viability studies demonstrated doxorubicin-loaded nanophores have a lower IC50 than free doxorubicin.

FIGS. 7A-7E depict the in vivo toxicity profile of doxorubicin-loaded Ferumoxytol and the therapeutic potential of dextran-nanophores in vivo.

FIG. 7A depicts the effect of drug-loaded ferumoxytol on creatinine levels.

FIG. 7B depicts the effect of drug-loaded ferumoxytol on the ratio of concentrations of aspartate transaminase (AST) and alanine transaminase (ALT) in the blood. The AST/ALT ratio is associated with liver damage or hepatotoxicity.

FIG. 7C depicts the effect of drug-loaded ferumoxytol on bilirubin levels.

FIG. 7D depicts the effect of drug-loaded ferumoxytol on sodium levels.

FIG. 7E depicts the effect of drug-loaded ferumoxytol on potassium levels. Chemotherapy with drug-loaded Ferumoxytol did not cause any toxicity to mice undergoing acute chemotherapy.

FIG. 8 depicts tumor growth response after clodrosome-based therapy.

FIGS. 9A-9D depict dextran-forming nanoparticles that are capable of retaining and delivering chemotherapeutics.

FIG. 9A depicts size distribution of unloaded (NP) and doxorubicin-loaded (Doxo-NP) dextran nanoparticles, determined with dynamic light scattering.

FIG. 9B depicts Fluorescence emission profiles of unloaded (NP) and doxorubicin-loaded (Doxo-NP) dextran nanoparticles (λ_{cx}=485 nm, λ_{cm}=590 nm).

FIG. 9C depicts doxorubicin-loaded dextran nanoparticles stably retained their cargo up to pH 7.0, but they released the drug at slightly acidic conditions (pH 6.8). Within˜1.5 hrs, 50% of the drug was released at this pH, suggesting that the nanoparticles could release their therapeutic payload at disease-relevant conditions.

FIG. 9D depicts doxorubicin delivered with dextran nanoparticles was more potent than the drug administered in its free form, since 2.5 μM doxorubicin delivered with the nanoparticles caused 50% reduction in the viability of PC3 cells as opposed to 8 μM of the free drug (mean±s.e.m.).

FIG. 10 depicts nanophores for the prevention of drug overdose. Dextran (left panel) and Feraheme (right panel) nanoparticles scavenged macromolecules, such as the fluorophore DiI, from the solution (mean±s.e.m.).

FIGS. 11A-11B depicts the ITLC spectrogram of the ⁸⁹Zr-carrying NPs showing complete radionuclide chelation in A) distilled water and B) phosphate-buffered saline (1×PBS).

FIG. 11C depicts a PET-CT scan after lh post-intradermal administration of the 89Zr-NPs in the footpad, allowing imaging of regional and distal lymphatic vessel drainage.

FIG. 11D shows the biodistribution the ⁸⁹Zr-NPs one hour post-intradermal administration, showing retention by the lymph nodes, and the nanoparticles' hepatic clearance.

Figures are presented herein for illustration purposes only, not for limitation.

### DEFINITIONS

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within

25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration”: The term “administration” refers to introducing a substance into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments. In some embodiments, administration is oral. Additionally or alternatively, in some embodiments, administration is parenteral. In some embodiments, administration is intravenous.

“Agent”: The term “agent” refers to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized include small molecules, antibodies, antibody fragments, aptamers, siRNAs, shRNAs, DNA/RNA hybrids, antisense oligonucleotides, ribozymes, peptides, peptide mimetics, peptide nucleic acids, small molecules, etc. In some embodiments, an agent is or comprises a polymer. In some embodiments, an agent contains at least one polymeric moiety. In some embodiments, an agent comprises a therapeutic, diagnostic and/or drug.

“Associated”: As used herein, the term “associated” typically refers to two or more entities in physical proximity with one another, either directly or indirectly (e.g., via one or more additional entities that serve as a linking agent), to form a structure that is sufficiently stable so that the entities remain in physical proximity under relevant conditions, e.g., physiological conditions. In some embodiments, associated moieties are covalently linked to one another. In some embodiments, associated entities are non-covalently linked. In some embodiments, associated entities are linked to one another by specific non-covalent interactions (e.g., by interactions between interacting ligands that discriminate between their interaction partner and other entities present in the context of use, such as, for example. streptavidin/avidin interactions, antibody/antigen interactions, etc.). Alternatively or additionally, a sufficient number of weaker non-covalent interactions can provide sufficient stability for moieties to remain associated. Exemplary non-covalent interactions include, but are not limited to, electrostatic interactions, hydrogen bonding, affinity, metal coordination, physical adsorption, host-guest interactions, hydrophobic interactions, pi stacking interactions, van der Waals interactions, magnetic interactions, electrostatic interactions, dipole-dipole interactions, etc.

“Biocompatible”: The term “biocompatible”, as used herein is intended to describe materials that do not elicit a substantial detrimental response in vivo. In certain embodiments, the materials are “biocompatible” if they are not toxic to cells. In certain embodiments, materials are “biocompatible” if their addition to cells in vitro results in less than

or equal to 20% cell death, and/or their administration in vivo does not induce inflammation or other such adverse effects. In certain embodiments, materials are biodegradable.

“Biodegradable”: As used herein, “biodegradable” materials are those that, when introduced into cells, are broken down by cellular machinery (e.g., enzymatic degradation) or by hydrolysis into components that cells can either reuse or dispose of without significant toxic effects on the cells. In certain embodiments, components generated by breakdown of a biodegradable material do not induce inflammation and/or other adverse effects in vivo. In some embodiments, biodegradable materials are enzymatically broken down. Alternatively or additionally, in some embodiments, biodegradable materials are broken down by hydrolysis. In some embodiments, biodegradable polymeric materials break down into their component polymers. In some embodiments, breakdown of biodegradable materials (including, for example, biodegradable polymeric materials) includes hydrolysis of ester bonds. In some embodiments, breakdown of materials (including, for example, biodegradable polymeric materials) includes cleavage of urethane linkages.

“Carrier”: As used herein, “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin

“Combination Therapy”: As used herein, the term “combination therapy”, refers to those situations in which two or more different pharmaceutical agents for the treatment of disease are administered in overlapping regimens so that the subject is simultaneously exposed to at least two agents. In some embodiments, the different agents are administered simultaneously. In some embodiments, the administration of one agent overlaps the administration of at least one other agent. In some embodiments, the different agents are administered sequentially such that the agents have simultaneous biologically activity with in a subject.

“Imaging Agent”: The term “imaging agent” as used herein refers to any element, molecule, functional group, compound, fragments thereof or moiety that facilitates detection of an agent (e.g., a polysaccharide nanoparticle) to which it is joined. Examples of imaging agents include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc. ), fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (e.g., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available.

“Pharmaceutically acceptable”: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

“Pharmaceutical composition”: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

“Physiological conditions”: The phrase “physiological conditions”, as used herein, relates to the range of chemical (e.g., pH, ionic strength) and biochemical (e.g., enzyme concentrations) conditions likely to be encountered in the intracellular and extracellular fluids of tissues. For most tissues, the physiological pH ranges from about 7.0 to 7.4.

“Polysaccharide”: The term “polysaccharide” refers to a polymer of sugars. Typically, a polysaccharide comprises at least three sugars. In some embodiments, a polysaccharide comprises dextran, amylose, amylopectin, glycogen, cellulose, arabonixylan, and/or pectin. In some embodiments, a polysaccharide comprises natural sugars (e.g., glucose, fructose, galactose, mannose, arabinose, ribose, and xylose); alternatively or additionally, in some embodiments, a polysaccharide comprises one or more non-natural amino acids (e.g, modified sugars such as 2′-fluororibose, 2′-deoxyribose, and hexose). In some embodiments, polysaccharide refers to dextran, a complex, branched glucan (composed of chains of varying lengths—from 3 to 2000 kD).

“Protein”: As used herein, the term “protein” refers to a polypeptide (e.g., a string of at least 3-5 amino acids linked to one another by peptide bonds). Proteins may include moieties other than amino acids (e.g., may be glycoproteins, proteoglycans, etc.) and/or may be otherwise processed or modified. In some embodiments “protein” can be a complete polypeptide as produced by and/or active in a cell (with or without a signal sequence); in some embodiments, a “protein” is or comprises a characteristic portion such as a polypeptide as produced by and/or active in a cell. In some embodiments, a protein includes more than one polypeptide chain. For example, polypeptide chains may be linked by one or more disulfide bonds or associated by other means. In some embodiments, proteins or polypeptides as described herein may contain L-amino acids, D-amino acids, or both, and/or may contain any of a variety of amino acid modifications or analogs known in the art. Useful modifications include, e.g., terminal acetylation, amidation, methylation, etc. In some embodiments, proteins or polypeptides may comprise natural amino acids, non-natural amino acids, synthetic amino acids, and/or combinations thereof. In some embodiments, proteins are or comprise antibodies, antibody polypeptides, antibody fragments, biologically active portions thereof, and/or characteristic portions thereof.

“Substantially”: As used herein, the term “substantially”, and grammatic equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

“Subject”: As used herein, the term “subject” includes humans and mammals (e.g., mice, rats, pigs, cats, dogs, and horses). In many embodiments, subjects are be mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be , for example, rodents (e.g., mice, rats, hamsters), rabbits, primates, or swine such as inbred pigs and the like.

“Therapeutic agent”: As used herein, the phrase “therapeutic agent” refers to any agent that has a therapeutic effect and/or elicits a desired biological and/or pharmacological effect, when administered to a subject.

“Treatment”: As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

“Unaltered imaging agent”: As used herein, the term “unaltered imaging agent” refers to any imaging agent that has not been chemically altered from one or more of its known forms.

“Unaltered therapeutic agent”: As used herein, the term “unaltered therapeutic agent” refers to any therapeutic that has not been chemically altered from one or more of its known forms (e.g., a known drug, e.g., a regulatory agency approved drug, e.g., an FDA approved drug).

### DETAILED DESCRIPTION

It is contemplated that compositions, systems, devices, methods, and processes of the claimed invention encompass variations and adaptations developed using information from the embodiments described herein. Adaptation and/or modification of the compositions, systems, devices, methods, and processes described herein may be performed by those of ordinary skill in the relevant art.

Throughout the description, where compositions, articles, and devices are described as having, including, or comprising specific components, or where processes and methods are described as having, including, or comprising specific steps, it is contemplated that, additionally, there are compositions, articles, and devices of the present invention that consist essentially of, or consist of, the recited components, and that there are processes and methods according to the present invention that consist essentially of, or consist of, the recited processing steps.

Similarly, where compositions, articles, and devices are described as having, including, or comprising specific compounds and/or materials, it is contemplated that, additionally, there are compositions, articles, and devices of the present invention that consist essentially of, or consist of, the recited compounds and/or materials.

It should be understood that the order of steps or order for performing certain action is immaterial so long as the invention remains operable. Moreover, two or more steps or actions may be conducted simultaneously.

The mention herein of any publication is not an admission that the publication serves as prior art with respect to any of the claims presented herein. Headers are provided for organizational purposes and are not meant to be limiting.

In certain embodiments, a new class of polysaccharide nanoparticle is presented herein for retaining and delivering unaltered therapeutic agents to treat diseases, disorders or conditions. Dextran-based nanoparticles effectively associate with unaltered therapeutic agents without any chemical modification to the agent. These agents associate with the nanoparticle in a non-covalent manner, through weak electrostatic, hydrogen bonding or van der Waals forces. Upon introduction to a subject, changes in the tissue microenvironments drive the release of agents from the nanoparticle complex. This causes the agents to be delivered at sites of diseases or disorders within the subject's body.

### Polysaccharide Nanoparticle Compositions

Polysaccharide nanoparticles described herein may be made of polysaccharides such as dextran, amylose, amylopectin, glycogen, cellulose, arabonixylan, and/or pectin. In certain embodiments, the polysaccharide is a dextran. Dextran is a complex, branched glucan (a polysaccharide made of many glucose molecules) composed of chains of varying lengths (from 3-2000 kilodaltons). The straight chain comprises alpha-1,6 glycosidic linkages between glucose molecules, while branching begins at alpha-1,3 linkages. In some embodiments, dextran nanoparticles are comprised of carboxymethyl dextran.

Polysaccharides that make up the nanoparticles (or nanoparticle surfaces) described herein can have a range of molecular weights. the polysaccharide has a molecular weight within a range of 1 kDa to 1 million kDa (e.g., 1-10 kDa, 10-100 kDa, 100-1000 kDa, or 1000-1,000,000 kDa). In some embodiments, the polysaccharide is dextran, amylose, amylopectin, glycogen, cellulose, arabonixylan, pectin, or some combination of two or more of these.

Polysaccharide nanoparticles described herein can have a range of sizes. In some embodiments, the nanoparticles have an average diameter in a range of 1 nm-500 nm (e.g., 1-10 nm, 10-25 nm, 25-50 nm, 50-100 nm, or 100-500 nm).

Polysaccharide nanoparticles described herein can be used in different uniformities. In some embodiments, polysaccharide nanoparticles may be relatively monodisperse (e.g., diameters of particles all within a range of 10 nm or less of each other). In other embodiments, the polysaccharide nanoparticles are more polydisperse.

### Mechanism of Retention and Delivery of Agents

Polysaccharide nanoparticles described herein are able to retain unaltered therapeutic agents without any chemical modification. The agents are retained by the nanoparticle vehicle through non-covalent interactions. These interactions include weak electrostatics, hydrogen bonding, and van der Waals forces. The agents are released (or delivered) at sites of diseases, disorders or conditions due to changes in the microenvironment. In some embodiments, microenvironmental changes that drive release of agents include changes in: acidity, osmolarity, and ionic strength.

In some embodiments, the agents are delivered to sites of disease (e.g., cancer/tumors) due to the EPR (Enhanced Permeability Retention) effect. The EPR effect is the property where molecules of certain sizes (for example: nanoparticles, macromolecular drugs, and liposomes) accumulate in tumor tissues at a higher rate than normal tissues. Tumor tissues often possess structural abnormalities that lead to greater permeability and also greater accumulation of circulating macromolecules. In general, non-tumor tissues with abnormal permeabilities could also experience greater accumulation of macromolecules (such as therapeutic agents). In some embodiments, polysaccharide nanoparticles described herein deliver agents to sites of diseases, disorders, and conditions with tissues that have abnormal cellular permeability.

### Diseases, Disorders, and Conditions

Polysaccharide nanoparticles described herein deliver unaltered therapeutic agents to sites of diseases, disorders, or conditions. The agents are released at sites where the condition has perturbed the local microenvironment enough to cause changes that disrupt the weak interactions between the therapeutic agent and the polysaccharide nanoparticle. Changes to the microenvironment which drive release of the agent include changes in: pH, osmolarity, and ionic strength.

Any disease or condition with changes to the microenvironment are susceptible to treatment with the nanoparticles described herein. Diseases such as cancer, rheumatoid arthritis, atherosclerosis, cardiac arrest, cystic fibrosis, diabetic ketoacidosis, stroke, renal failure, malaria, lactic acid acidosis, and inflammatory conditions and disorders may be treatable by polysaccharide nanoparticles described herein.

In some embodiments, the disease, disorder or condition treated by the polysaccharide nanoparticles is cancer. Cancers that are treated include: prostate cancer, breast cancer, testicular cancer, cervical cancer, lung cancer, colon cancer, bone cancer, glioma, glioblastoma, multiple myeloma, sarcoma, small cell carcinoma, renal cancer, liver cancer, head and neck cancer, esophageal cancer, thyroid cancer, lymphoma, and leukemia.

In some embodiments, polysaccharide nanoparticles are used in combination with treatments comprising antibodies, small molecule drugs, radiation, pharmacotherapy, chemotherapy, cryotherapy, thermotherapy, electrotherapy, phototherapy, ultrasonic therapy and surgery.

### Therapeutic Agents

A wide variety of unaltered therapeutic agents can be used for delivery by polysaccharide nanoparticles described herein. In some embodiments, therapeutic agents comprise chemotherapeutic drugs. Chemotherapeutic drugs used as agents include, but are not limited to: doxorubicin, amphotericin B, daunarubicine, cytarabine, Xtandi, MDV3100, PI3K inhibitors, BEZ235, MEK inhibitors, AZD6244, Selumetinib®, EGFR inhibitors, enzalutamide, methotrexate, cytarabine, gemcitabine, decitabine, Vidaza, fludarabine, nelarabine, cladribine, clofarabine, pentostatin, thioguanine, mercaptopurine, Afatinib, Axitinib, Bevacizumab, Bosutinib, Cetuximab, Crizotinib, Dasatinib, Erlotinib, Fostamatinib, Gefitinib, Ibrutinib, Imatinib, Lapatinib, Lenvatinib, Mubritinib, Nilotinib, Panitumumab, Pazopanib, Pegaptanib, Ranibizumab, Sunitinib, SU6656, Trastuzumab, Tofacitinib, Vandetanib, Vemurafenib, and kinase inhibitors.

In certain embodiments, the compositions described herein include (i) imaging agents that are, or are associated with, the therapeutic agent, and/or (ii) imaging agents that are associated with, or are a part of, the nanoparticles. In some embodiments, the imaging agents can include radiolabel s, radionuclides, radioisotopes, fluorophores, fluorochromes, dyes, metal lanthanides, paramagnetic metal ions, superparamagnetic metal oxides, ultrasound reporters, x-ray reporters, and/or fluorescent proteins.

In some embodiments, radiolabels comprise ^{99m}TC, ¹¹¹In, ⁶⁴Cu, ⁶⁷Ga, ¹⁸⁶Re, ¹⁸⁸Re, ¹⁵³Sm, ¹⁷⁷Lu, ⁶⁷Cu, ¹²³I, ¹²⁴I, ¹²⁵I, ¹¹C, ¹3N, ¹⁵O, ¹⁸F, ¹⁸⁶Re, ¹⁸⁸Re, ¹⁵³Sm, ¹⁶⁶Ho, ¹⁷⁷Lu, ¹⁴⁹Pm, ⁹⁰Y, ²¹²Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ⁶⁷Cu, ¹⁰⁵Rh, ¹¹¹Ag, ⁸⁹Zr, and ¹⁹²Ir. In some embodiments, paramagnetic metal ions comprise Gd(III), Dy(III), Fe(III), and Mn(II). In some embodiments, Gadolinium (III) contrast agents comprise Dotarem, Gadavist, Magnevist, Omni scan, OptiMARK, and Prohance. In some embodiments, x-ray reporters comprise iodinated organic molecules or chelates of heavy metal ions of atomic numbers 57 to 83.

In some embodiments, PET (Positron Emission Tomography) tracers are used as imaging agents. In some embodiments, PET tracers comprise ⁸⁹Zr, ⁶⁴Cu, [¹⁸F] fluorodeoxyglucose.

In some embodiments, fluorophores comprise fluorochromes, fluorochrome quencher molecules, any organic or inorganic dyes, metal chelates, or any fluorescent enzyme substrates, including protease activatable enzyme substrates. In some embodiments, fluorophores comprise long chain carbophilic cyanines. In other embodiments, fluorophores comprise DiI, DiR, DiD, and the like. Fluorochromes comprise far red, and near infrared fluorochromes (NIRF) Fluorochromes include but are not limited to a carbocyanine and indocyanine fluorochromes. In some embodiments, imaging agents comprise commercially available fluorochromes including, but not limited to Cy5.5, Cy5 and Cy7 (GE Healthcare); AlexaFlour660, AlexaFlour680, AlexaFluor750, and AlexaFluor790 (Invitrogen); VivoTag680, VivoTag-S680, and VivoTag-S750 (VisEn Medical); Dy677, Dy682, Dy752 and Dy780 (Dyomics); DyLight547, DyLight647 (Pierce); HiLyte Fluor 647, HiLyte Fluor 680, and HiLyte Fluor 750 (AnaSpec); IRDye 800CW, IRDye 800RS, and IRDye 700DX (Li-Cor); and ADS780WS, ADS830WS, and ADS832WS (American Dye Source) and Kodak X-SIGHT 650, Kodak X-SIGHT 691, Kodak X-SIGHT 751 (Carestream Health).

### Administration

Pharmaceutical compositions incorporating the polysaccharide nanoparticles described herein may be administered according to any appropriate route and regimen. In some embodiments, a route or regimen is one that has been correlated with a positive therapeutic benefit.

In some embodiments, the exact amount administered may vary from subject to subject, depending on one or more factors as is well known in the medical arts. Such factors may include, for example, one or more of species, age, general condition of the subject, the particular composition to be administered, its mode of administration, its mode of activity, the severity of disease; the activity of the specific polysaccharide nanoparticles employed; the specific pharmaceutical composition administered; the half-life of the composition after administration; the age, body weight, general health, sex, and diet of the subject; the time of administration, route of administration, and rate of excretion of the specific compound employed; the duration of the treatment; drugs used in combination or coincidental with the specific compound employed and the like. Pharmaceutical compositions may be formulated in dosage unit form for ease of administration and uniformity of dosage. It will be understood, however, that the total daily usage of the compositions will be decided by an attending physician within the scope of sound medical judgment.

Compositions described herein may be administered by any route, as will be appreciated by those skilled in the art. In some embodiments, compositions described herein are administered by oral (PO), intravenous (IV), intramuscular (IM), intra-arterial, intramedullary, intrathecal, subcutaneous (SQ), intraventricular, transdermal, interdermal, intradermal, rectal (PR), vaginal, intraperitoneal (IP), intragastric (IG), topical (e.g., by powders, ointments, creams, gels, lotions, and/or drops), mucosal, intranasal, buccal, enteral, vitreal, sublingual; by intratracheal instillation, bronchial instillation, and/or inhalation; as an oral spray, nasal spray, and/or aerosol, and/or through a portal vein catheter.

In some embodiments, the pharmaceutical compositions and/or polysaccharide nanoparticles thereof may be administered intravenously (e.g., by intravenous infusion), by intramuscular injection, by intratumoural injection, and/or via portal vein catheter, for example. However, the subject matter described herein encompasses the delivery of pharmaceutical compositions and/or polysaccharide nanoparticles thereof in accordance with embodiments described herein by any appropriate route taking into consideration likely advances in the sciences of drug delivery.

In some embodiments, the pharmaceutical compositions and/or polysaccharide nanoparticles thereof may be administered at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg of subject body weight per day to obtain the desired therapeutic effect. The desired dosage may be delivered more than three times per day, three times per day, two times per day, once per day, every other day, every third day, every week, every two weeks, every three weeks, every four weeks, every two months, every six months, or every twelve months. In some embodiments, the desired dosage may be delivered using multiple administrations (e.g., two, three, four, five, six, seven, eight, nine, ten, eleven, twelve, thirteen, fourteen, or more administrations).

### Prophylactic Applications

In some embodiments, compositions described herein may be utilized for prophylactic applications. In some embodiments, prophylactic applications involve systems and methods for preventing, inhibiting progression of, and/or delaying the onset of cancer or other disorder, and/or any other gene-associated condition in individuals susceptible to and/or displaying symptoms of cancer or other disorder.

### Combination Therapy

It will be appreciated that pharmaceutical compositions described herein can be employed in combination therapies to aid in diagnosis and/or treatment. “In combination” is not intended to imply that the agents must be administered at the same time and/or formulated for delivery together, although these methods of delivery are within the scope of the embodiments described herein. Compositions can be administered concurrently with, prior to, or subsequent to, one or more other desired therapeutics or medical procedures. In will be appreciated that therapeutically active agents utilized in combination may be administered together in a single composition or administered separately in different compositions. In general, each agent will be administered at a dose and/or on a time schedule determined for that agent.

The particular combination of therapies (e.g., therapeutics or procedures) to employ in a combination regimen will take into account compatibility of the desired therapeutics and/or procedures and the desired therapeutic effect to be achieved. It will also be appreciated that pharmaceutical compositions of the polysaccharide nanoparticles disclosed herein can be employed in combination therapies (e.g., combination chemotherapeutic therapies), that is, the pharmaceutical compositions can be administered concurrently with, prior to, or subsequent to, one or more other desired therapeutic and/or chemotherapeutic procedures.

The particular combination of therapies to employ in a combination regimen will generally take into account compatibility of the desired therapeutics and/or procedures and the desired therapeutic effect to be achieved. It will also be appreciated that the therapies and/or chemotherapeutics employed may achieve a desired effect for the same disorder (for example, an inventive antigen may be administered concurrently with another chemotherapeutic or neurological drug), or they may achieve different effects. It will be appreciated that the therapies employed may achieve a desired effect for the same purpose (for example, polysaccharide nanoparticles useful for treating, preventing, and/or delaying the onset of cancer or other disorder may be administered concurrently with another agent useful for treating, preventing, and/or delaying the onset of cancer or disorders), or they may achieve different effects (e.g., control of any adverse effects). The subject matter described herein encompasses the delivery of pharmaceutical compositions in combination with agents that may improve their bioavailability, reduce and/or modify their metabolism, inhibit their excretion, and/or modify their distribution within the body.

In some embodiments, agents utilized in combination will be utilized at levels that do not exceed the levels at which they are utilized individually. In some embodiments, the levels utilized in combination will be lower than those utilized individually.

### EXPERIMENTAL EXAMPLES

### Example 1

### Retention of Cargo by Polysaccharide Nanoparticles

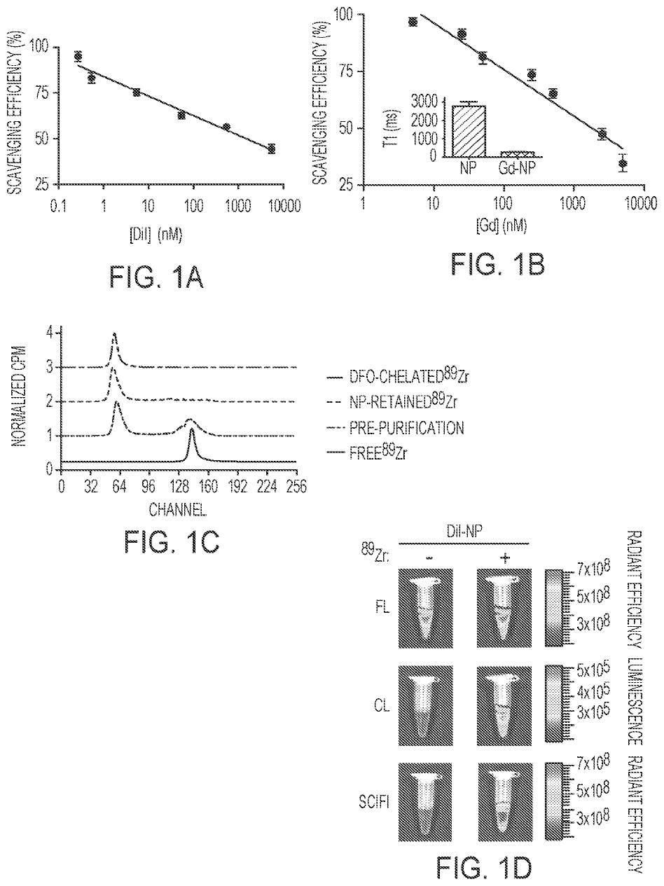

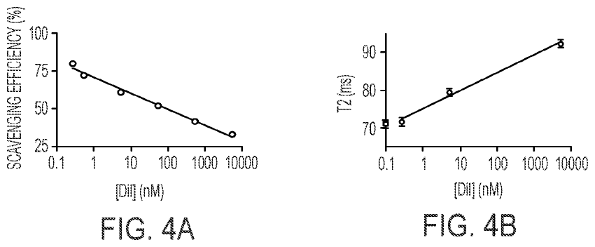

This example demonstrates that nascent dextran nanophores can retain cargo similar to their iron oxide counterparts. Solutions were prepared with different concentrations of a prototypic hydrophobic high-molecular-weight cargo in a mixed solvent system. Under continuous gentle stirring, a fixed concentration of nanoparticles was added drop-wise into the bulk solution, containing the fluorophore DiI. After purification to remove any unbound cargo, the nanoparticles' retention (scavenging) efficiency was determined spectrophotometrically, by comparing the absorbance/fluorescence emission of the nanoparticle-scavenging and original solutions (FIG. 1A). When comparing equal scavenging concentrations of dextran and Ferumoxytol nanophores, the dextran nanoparticles more effectively sequestered DiI than Ferumoxytol (FIG. 4A). After scavenging the dye DiI from the solution, the magnetic properties of Feraheme nanoparticles were examined with a compact relaxometer. The entrapped dye molecules increased the nanoparticles' T2 relaxation times when compared to the unloaded nanoparticles (green square; mean±s.e.m.). The sequestration of a macromolecule from the solution affects the magnetic properties of Feraheme nanophores. This might be attributed to dextran's thicker polymer layer and the ability of cargo to more extensively associate with it via weak electrostatic interactions. Functional groups involved in cargo retention might be utilized for the association of the carboxymethyl dextran with the iron oxide core, rendering Ferumoxytol a less efficient cargo carrier. Cargo retention by Ferumoxytol affected the formulation's magnetic properties, reflected in increased T2 signal, due to the cargo hindering the diffusion of water molecules within the nanoparticle coating (FIG. 4B). This example demonstrates that polysaccharide-based nanoparticles can retain their cargo in a manner more efficient than iron oxide nanoparticles.

The ability of dextran nanophores to retain radiological tracers for magnetic resonance imaging (MRI) and positron emission tomography (PET) was investigated. The nanophores sequestered gadolinium from the solution, and stably retained it after filtration, which yielded decreases in the nanophore's T1 signal (FIG. 1B). Dextran nanophores sequestered the positron emitter radionuclide ⁸⁹Zr (FIG. 1C), which has a half-life of 78.4 hours that is ideal for the tracking of long-circulating macromolecular and nanoparticle constructs. Multimodality is frequently desired in the clinical setting to facilitate pre-operative and (intra)operative imaging using a common imaging agent. The light generated during the decay of radionuclides, such as ⁸⁹Zr, that occurs due to the charged particle's high speed that exceeds the speed of light in a dielectric medium (Cherenkov luminescence, CL) was utilized for this purpose. Co-retention of DiI and ⁸⁹Zr allowed excitation of the fluorophore with an external source (FL) and its own radionuclide, where the broad-spectrum of the Cherenkov luminescence light excites a fluorescent dye (secondary Cherenkov-induced fluorescence, SCIFI) (FIG.. 1D). Nanophores loaded only with DiI fluoresced solely upon excitation by the imaging instrument's light source, and lacked any Chereknov luminescence. Collectively, these data show that polysaccharide (e.g., dextran) nanophores can serve as multimodal carriers of clinically relevant tracers, and allow tracking of the nanoparticles through clinical imaging readers prior or during surgery, plausibly providing important decision-making information like lymph node metastasis, vascularization and perfusion, among others.

### Example 2

### Release of Cargo by Polysaccharide Nanoparticles

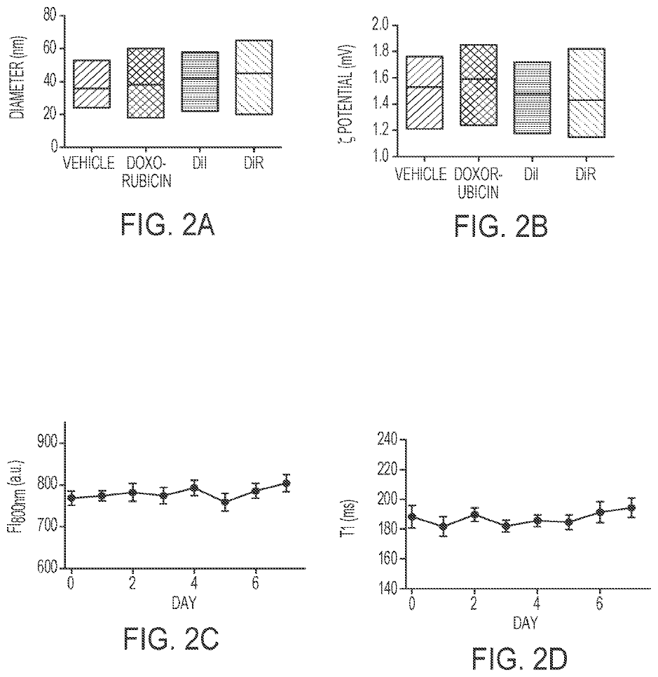

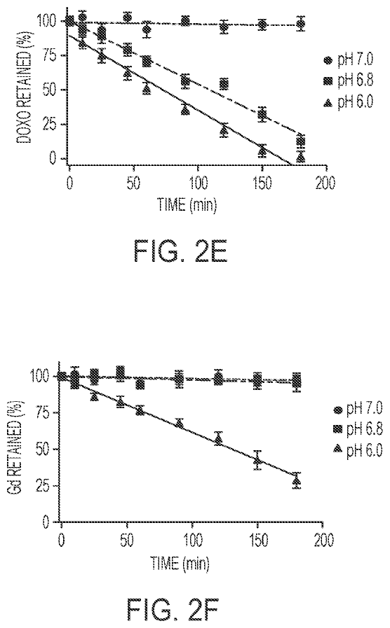

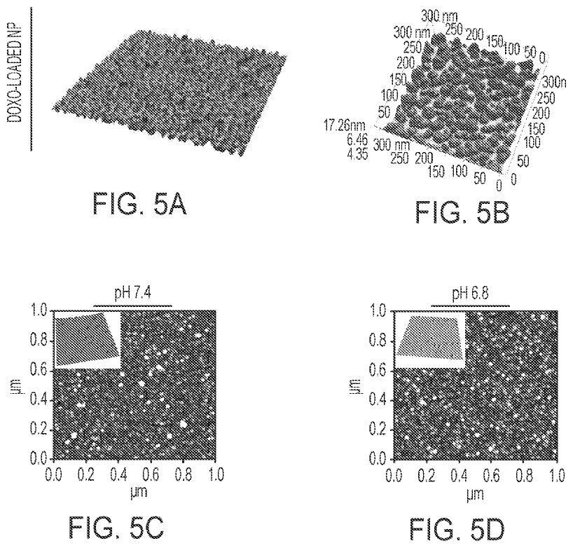

Next cargo retention and encapsulation, as well as release, was investigated for any effect on the structure of dextran nanophores. Following loading of the nanophores via the solvent diffusion method and purification through dialysis, the nanoparticles' size was determined using dynamic light scattering. Loading of cargo neither affected the size (FIG. 2A) nor the surface charge (FIG. 2B) of the nanoparticles, likely due to retention of the molecular payload within the polymer's internal cavities that provide an extensive network of weak electrostatic associations, such as hydrogen bonds and van der Waals forces, leaving the polymer's surface groups to interact with water molecules comprising the nanoparticle solvation sphere. The serum stability of the nanophores was determined using nanoparticles loaded with either the hydrophobic near-infrared fluorophore DiR or gadolinium ions. During the course of a week, no changes in the nanoparticles fluorescence and magnetic signal were detected (FIG. 2C-2D), demonstrating that the nanoparticles can stably retain their cargo at physiological conditions. However, release of the cargo occurred at different conditions after acidification of the aquatic milieu. First, doxorubicin-carrying nanophores rapidly released the drug once the pH dropped below 7.0, such as pH 6.8 that is encountered in many solid tumors (FIG. 2E). Secondly, doxorubicin was released in a slightly faster rate at pH 6.0 than pH 6.8, perhaps due to the wider perturbation of the forces holding together the drug with the nanoparticle at this pH level. In the case of gadolinium, the nanophores retained it at pH 6.8 but released it at pH 6.0(FIG. 2F), perhaps due to the higher oxygen philicity of the radiological tracer. Atomic force microscopy confirmed that the nanoparticles preserved their size after microenvironment-driven cargo release (FIGS. 5A-5D), further supporting the notion that the cargo loading and release processes do not affect the vehicle's physical characteristics, hence its pharmacokinetics properties remain unaltered. This example demonstrates that polysaccharide (e.g., dextran) nanoparticles can retain and release cargo without affecting the nanoparticle itself.

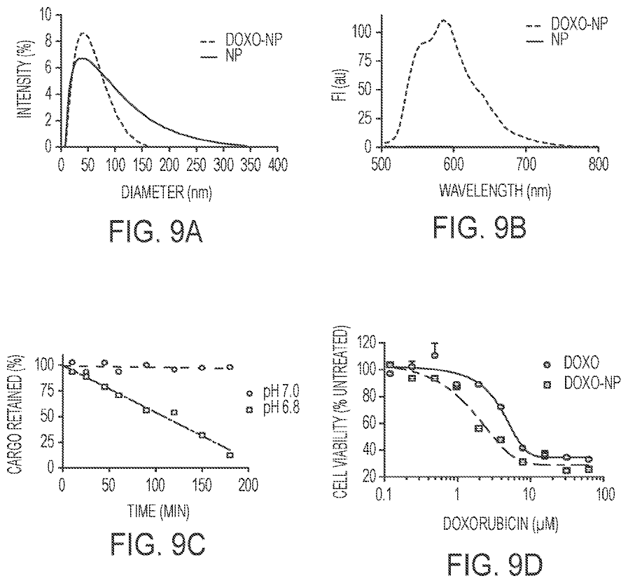

The ability of dextran to form nanoparticles capable of retaining and delivering chemotherapeutics was also investigated. The size distribution of unloaded (NP) and doxorubicin-loaded (Doxo-NP) dextran nanoparticles was determined with dynamic light scattering (see FIG. 9A). Fluorescence emission profiles of unloaded (NP) and doxorubicin-loaded (Doxo-NP) dextran nanoparticles (λₑₓ=485 nm, λₑₘ=590 nm) were generated as in FIG.

9B. The doxorubicin-loaded dextran nanoparticles stably retained their cargo up to pH 7.0, but they released the drug at slightly acidic conditions (pH 6.8

) (see FIG. 9C). Within ˜1.5 hrs, 50% of the drug was released at this pH, suggesting that the nanoparticles could release their therapeutic payload at disease-relevant conditions. Doxorubicin delivered with dextran nanoparticles was more potent than the drug administered in its free form (see FIG. 9D), since 2.5 μM doxorubicin delivered with the nanoparticles caused 50% reduction in the viability of PC3 cells as opposed to 8 μM of the free drug (mean±s.e.m.).

### Example 3

### Therapeutic Potential of Polysaccharide Nanoparticles

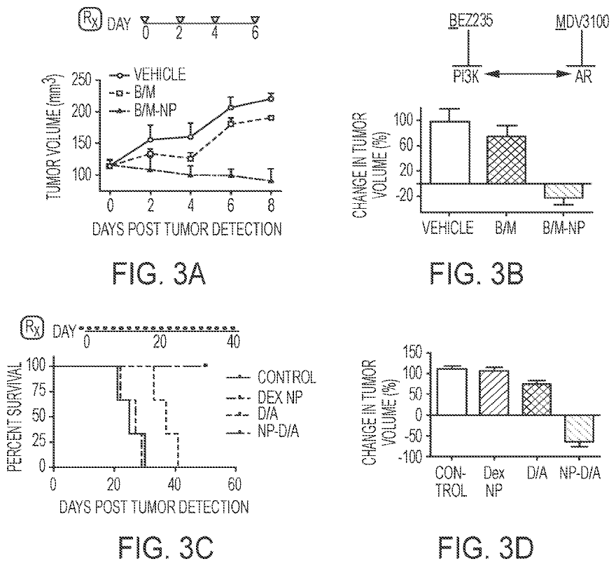

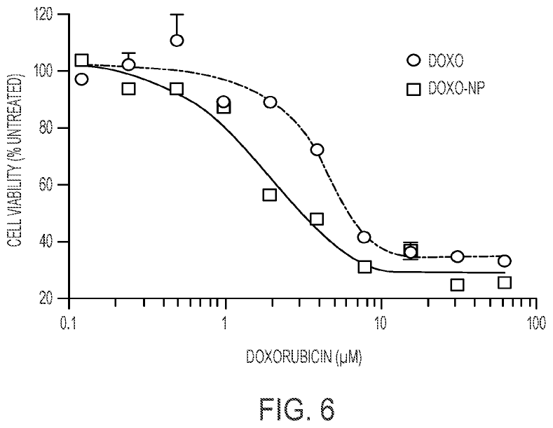

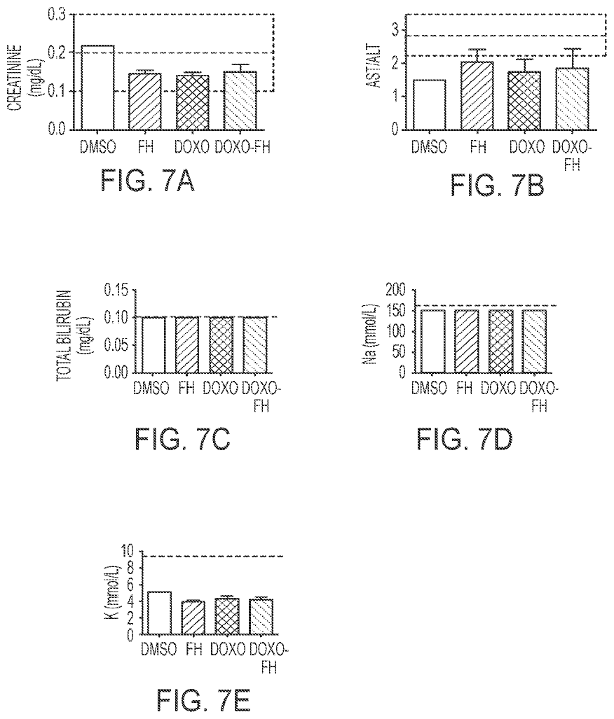

Subsequently, the dextran nanophores therapeutic potential in vitro and in vivo was investigated. Cell viability studies using the human prostatic adenocarcinoma cell line PC3 showed that the doxorubicin-loaded nanophores had a lower IC50 than free drug (3.1 μM vs 6.5 μM, FIG. 6). Although chemotherapy with drug-loaded Ferumoxytol did not cause any toxicity to mice undergoing acute chemotherapy (FIGS. 7A-7E), a potential translational limitation of the use of these nanoparticles as drug delivery vehicle in the clinic is the possibility of iron overloading. Hence, the use of dextran-based nanophores for combinatorial chemotherapy and the simultaneous delivery of drugs to concurrently inhibit major oncogenic pathways and cellular processes was examined. Studies with athymic male nude mice that had xenografts of the androgen-receptor-positive human prostatic adenocarcinoma cell line LNCaP showed that dextran nanophores co-loaded with the anti-androgen MDV3100 (Xtandi®) and the PI3K inhibitor BEZ235 were able to achieve tumor regression, as opposed to the free drugs that had no efficacy (FIGS. 3A-3B). This drug combination was selected, because it was previously reported that inhibition of the androgen receptor pathway leads to overactivation of the PI3K cascade, while suppression of PI3K upregulates androgen-receptor-mediated signaling. Apart from prostate cancer, nanophores was studied for use as combinatorial therapy of other tumors, including triple-negative breast cancer, where it was recently shown that treatment with doxorubicin and EGFR inhibitor led to enhanced cell death and tumor regression. Doxorubicin and the MEK inhibitor AZD6244 (Selumetinib®) was co-loaded into dextran nanophores, since MEK is a downstream target of EGFR. Long-term treatment of mice bearing human triple-negative breast cancer xenografts (MDA-MB-468) with the nanophores demonstrated improved survival and tumor regression (FIGS. 3C-3D), contrary to the free drugs. This demonstrates that polysaccharide (e.g., dextran) nanoparticles can carry and release drugs to sites of disease.

Other drug-loaded dextran nanoparticles also simultaneously deliver a combination of drugs for improved therapy. In vivo studies with male nude mice bearing human prostate cancer xenografts of the androgen-responsive LNCaP cell line demonstrated that simultaneous delivery of BKM120 (“B”) and enzalutamide (MDV3100 or “M”) in the same dextran nanoparticle (B/M-NP) caused tumor reduction (as depicted in FIG. 3A). Control animals were either treated with vehicle (DMSO) or combination of the free drugs (B/M) administered iv at the same concentration and dosing schedule like the nanoparticle-based delivery. The animals were treated on day 0, 2, 4 and 6, and all animals were euthanized on day 8 (n=3 per treatment group, mean±s.e.m.). At the end of the study (day 8 of the treatment regimen), the mice that were treated with the drug-loaded nanoparticles showed tumor reduction, as opposed to the animals that received the free drug combination that had tumor volumes comparable to the vehicle-treated animals (DMSO) (mean±s.e.m.) (see FIG. 3B). This demonstrates that dextran nanoparticles can deliver drugs to treat disease in live subjects.

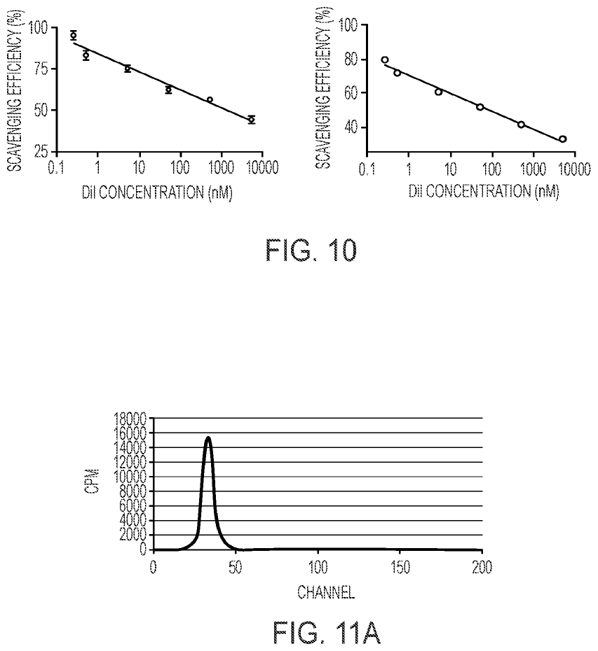

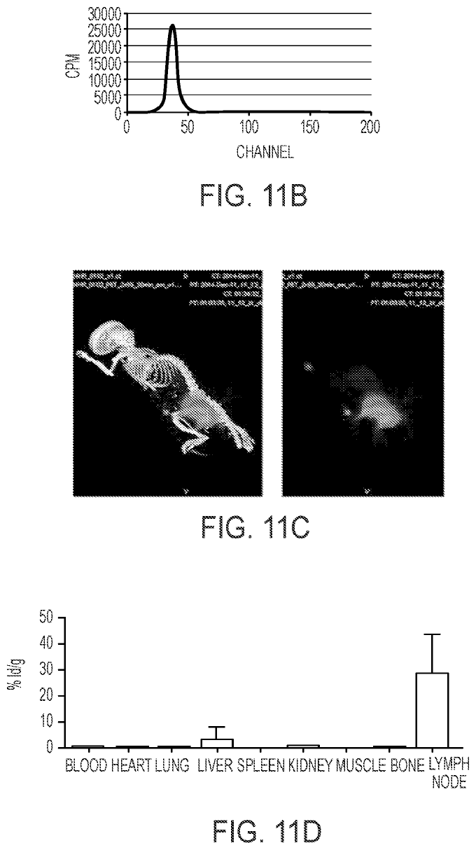

Due to their scavenging efficiency, polysaccharide nanoparticles can be used to prevent drug overdose. As shown in FIG. 10, dextran (left panel) and feraheme (right panel) nanoparticles were able to scavenge and retain macromolecules from solution, such as the fluorophore DiI. This demonstrates the ability of polysaccharide nanoparticles and iron oxide nanoparticles to treat or prevent drug overdosing.

### Example 4

### Mechanism of Polysaccharide Nanoparticle Therapy

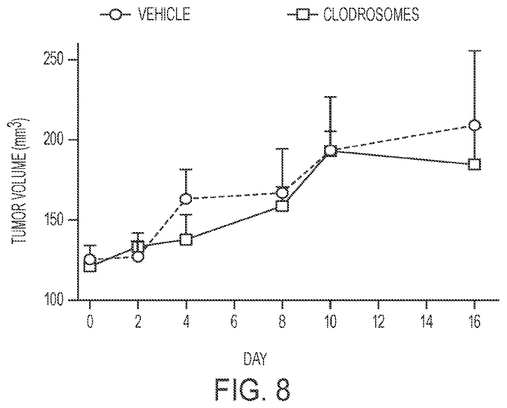

To exclude the possibility that the nanophores' therapeutic effect was due to uptaking by and death of tumor-associated macrophages, tumor-bearing animals were treated with liposome-encapsulated clodronate (Clodrosomes®) that targets macrophages and causes their depletion. Considering the findings that treatment with clodrosomes did not cause any tumor growth suppression and regression (FIG. 8), the drug-loaded nanophores primarily exert their therapeutic activity directly on the tumor through improved delivery and microenvironment-based release of their cargo. This example demonstrates that the therapeutic effect of polysaccharide (e.g., dextran) nanoparticles delivering drugs to sites of disease is independent of uptake by macrophages.

Polymeric dextran-based nanophores can be used for combinatorial therapy and chelation of medically relevant tracers. Since retention of the payload is mediated via weak electrostatic interactions that do not physically or chemically alter the cargo or the nanoparticles, these nanophores can serve as translational chemotherapeutic carriers, without receiving extensive scrutiny from regulatory agencies. With the emergence of new therapeutic interventions and novel imaging platforms for the operating room, these multifunctional platforms may improve clinical decision-making and patient care, by providing vital information, such as sentinel lymph node drainage and metastasis. The translation of these polymeric nanophores to areas other than oncology, such as infectious disease and inflammatory syndromes, is anticipated, opening new nanoscale-based therapeutic venues.

### EQUIVALENTS

While the invention has been particularly shown and described with reference to specific preferred embodiments, it should be understood by those skilled in the art that various changes in form and detail may be made therein without departing from the spirit and scope of the invention as defined by the appended claims.

1. A method of delivering one or more agents to a disease site, an infection site, an inflammation site, or an organ in a subject, the method comprising:
administering a composition comprising:
one or more unaltered agents associated with nanoparticles comprising dextran wherein the nanoparticles do not have a crystalline core and wherein an intensity-weighted average diameter of the nanoparticles as determined by dynamic light scattering is from 15 nm to 200 nm.

2. (canceled)

3. The method of claim 1, wherein the nanoparticles are at least 50 wt. % dextran.

4. The method of claim 1, wherein each of the nanoparticles have a surface comprising the dextran.

5. The method of claim 1, wherein the nanoparticles have an intensity-weighted average diameter as determined by dynamic light scattering of 25 nm to 100.

6. The method of claim 1, wherein the dextran has a molecular weight within a range of 1 kDa to 1 million kDa.

7. The method of claim 1, wherein the nanoparticles further comprise a member selected from the group consisting of amylose, amylopectin, glycogen, cellulose, arabonixylan, and pectin.

8. The method of claim 1, wherein the subject is suffering from a disease, disorder, or condition selected from the group consisting of cancer, rheumatoid arthritis, atherosclerosis, cystic fibrosis, diabetic ketoacidosis, cardiac arrest, stroke, renal failure, malaria, lactic acid acidosis, and inflammation.

9. The method of claim 8, wherein the disease, disorder, or condition is cancer.

10. The method of claim 9, wherein the cancer is a member selected from the group consisting of prostate cancer, breast cancer, brain cancer, testicular cancer, cervical cancer, lung cancer, colon cancer, glioma, glioblastoma, multiple myeloma, sarcoma, bone cancer, small cell carcinoma, renal cancer, liver cancer, head and neck cancer, esophageal cancer, thyroid cancer, lymphoma, and leukemia.

11. The method of claim 1, wherein the unaltered agent therapeutic is a chemotherapy drug.

12. The method of claim 11, wherein the chemotherapy drug is a member selected from the group consisting of doxorubicin, amphotericin B, daunarubicine, cytarabine, enzalutamide, methotrexate, cytarabine, gemcitabine, decitabine, azacitidine, fludarabine, nelarabine, cladribine, clofarabine, pentostatin, thioguanine, mercaptopurine, photosensitizer, biologic, including peptides and peptidomimetics, and kinase inhibitor.

13. The method of claim 11, wherein the chemotherapeutic drug is doxorubicin.

14. (canceled)

15. The method of claim 1, wherein the unaltered agent is an imaging agent.

16. The method of claim 1, wherein the one or more unaltered agents comprise at least one therapeutic agent and at least one imaging agent.

17. The method of claim 1, wherein the dextran comprises unsubsituted dextran, carboxymethyl dextran, or both unsubstituted dextran and carboxymethyl dextran.

18.-66. (canceled)
